(19) United States
(12) Patent Application Publication
(10) Pub. No.: US 2025/0008953 A1
(43) Pub. Date: Jan. 9, 2025

(54) METHOD AND USE OF PROTEIN-RICH MIXTURE

(71) Applicant: STAM AGRO NV, Dendermonde (BE)

(72) Inventor: Jef VANOVERSCHELDE, Dendermonde (BE)

(21) Appl. No.: 18/709,724

(22) PCT Filed: Nov. 17, 2022
(86) PCT No.: PCT/IB2022/061076

§ 371 Date: May 13, 2024

(30) Foreign Application Priority Data
Nov. 17, 2021 (BE) 2021/5887

(51) Int. Cl. A01N 37/46; A01P 21/00; C07K 1/34; C07K 14/465; C07K 14/47; C12N 9/48; C12N 9/50; C12P 21/06

(52) U.S. Cl. CPC A01N 37/46; A01P 21/00; C07K 1/34; C07K 14/465; C07K 14/47; C12N 9/485; C12N 9/50; C12P 21/06

(57) ABSTRACT

The present invention relates to a method of producing an enzymatically hydrolyzed mixture of feathers and/or hair. The invention also relates to a hydrolyzed mixture and its use for stimulating plant growth.

### TECHNICAL FIELD

The present invention relates to a method for producing an enzymatically hydrolyzed mixture of feathers and/or hair, comprising a step of enzymatically hydrolyzing, and the mixture resulting therefrom. The present invention further relates to the use of this mixture as a biostimulant.

### PRIOR ART

Industrial poultry farms generate large amounts of waste, such as feathers, which must be disposed of. Feathers and hair contain a high content of keratin (85-100%), a protein that is unfortunately indigestible for animals in its natural state due to its high degree of polymerization. Keratin contains sulfur bridges between two cysteine units. These disulfide bonds make the protein insoluble and must be broken to allow digestion.

Peptide bonds can be broken down by hydrolysis, which hydrolysis may be performed with water and physical means (pressure and heat), chemical means (1% hydrochloric acid solution, dimethylsulphoxide, sodium sulfide, sodium thioglycolate, sodium hydroxide), or with biological means (enzymes like bacterial or fungal keratinase, alkaline protease, or with specific microorganisms).

Whereas some hydrolytic processes might improve protein digestibility, they could, at the same time, reduce the nutritional value of the resulting product since changes in the amino acid pattern can occur.

BE1024444 describes a process for producing a hydrolyzable mixture from feathers and/or hair. The thermally hydrolyzed product obtained can be used as an agrochemical composition, an edible composition for animals or a cosmetic composition.

EP1241149 describes a method to make a fertilizer for plants via hydrolysis of proteins. More specifically, animal proteins derived from offal are used. By using protease enzymes, these proteins are broken down, thereby obtaining a mixture that can be used as a fertilizer for plants.

Calcium hydroxide is used to obtain the hydrolyzate. Fats are removed for example by centrifuge. The enzymatic hydrolysis is carried out in such a way that peptides with an average molecular mass between 500 and 1000 Da are obtained.

CZ24517 describes a hydrolyzate with a molecular weight of 3000 to 100000 g/mol. These molecules are relatively large and may be less easily absorbed by plants, for example.

WO2016110523 describes a hydrolyzate in which 80% of the hydrolyzate comprises peptides with a molecular weight of 100-800 Da.

The present invention aims at a suitable product with a more valuable application. The object of the invention is to provide a method from which this product can be obtained.

### SUMMARY OF THE INVENTION

In a first aspect, the present invention relates to a method according to claim 1. This method has the advantage, among other things, that a mixture of feathers and/or hair can be obtained in a low-energy manner with many peptides with a low molecular weight. Preferred embodiments of the method are described in dependent claim 2 up to and including 8.

In a second aspect, the present invention relates to a mixture according to claim 9. Preferred embodiments of the device are shown in claims 10 and 11.

In a third aspect the present invention relates to a use according to claim 12 and in a fourth aspect according to claim 13. The use of a thermally and/or enzymatically hydrolyzed mixture results in improved plant growth. In addition, the resistance of plants to biotic and abiotic stress improves after exposure to such a mixture.

The use of the thermally and/or enzymatically hydrolyzed mixture, even in low concentrations, reduces the need for traditional fertilization. With traditional fertilization, a large amount of nutrients is sprayed onto or injected into the soil. A large part of this leaches out, which leads to ecological problems. By spraying low amounts of the hydrolyzed mixture on the plants, the nitrogen load on the environment is reduced. In this way, nitrogen-polluted soils can slowly recover.

### Definitions

Unless otherwise defined, all terms used in the description of the invention, including technical and scientific terms, have the meaning generally understood by those skilled in the technical field of the invention. For a better understanding of the description of the invention, the following terms are explained explicitly.

In this document, “a”, “an” and “the” refer to both the singular and the plural referents, unless the context presupposes otherwise. For example, “a segment” means one or more than one segments.

When the term “around” or “about” is used in this document with a measurable quantity, a parameter, a duration or moment, and the like, then variations are meant of approx. 20% or less, preferably approx. 10% or less, more preferably approx. 5% or less, even more preferably approx. 1% or less, and even more preferably approx. 0.1% or less than and of the quoted value, insofar as such variations are applicable in the described invention. However, it must be understood that the value of a quantity used where the term “about” or “around” is used, is itself specifically disclosed.

The terms “comprise”, “comprising”, “consist of”, “consisting of”, “provided with”, “include”, “including”, “contain”, “containing” are synonyms and are inclusive or open terms that indicate the presence of what follows, and which do not exclude or prevent the presence of other components, features, elements, members, steps, as known from or disclosed in the prior art.

Quoting numerical ranges by endpoints includes all integers, fractions and/or real numbers between the endpoints, these endpoints included.

The expression “percent by weight,” “wt %” or “% by weight” in this document refers to the relative weight of a component based on the total weight of the entire referenced product.

The terms “molecular mass”, “molecular weight” and “molecule mass” are used synonymously herein.

A biostimulant is a substance applied to plants to increase nutrient efficiency, increase resistance to stress from biotic or abiotic factors, increase productivity and/or improve product quality. The biostimulant is applied in much lower quantities than fertilizers.

The use of a product as a biostimulant differs from the use of a product as a fertilizer in the amount of nutrients applied. For use as a biostimulant, the amount of nitrogen, phosphorus and potassium is less than 5% of the annual required amount, preferably less than 1%, more preferably less than 0.1%. For use as fertilizer, the amount of nitrogen, phosphorus or potassium is at least 20% of the annual required amount, preferably at least 40%.

### DETAILED DESCRIPTION

In a first aspect, the invention relates to a method for producing a hydrolyzed mixture of feathers and/or hair and/or skins, said method comprising:
providing a composition comprising feathers and/or hair and water, the composition having a dry matter content of less than 50 wt % with respect to the total weight of the composition;
pressing at least a portion of the water from the composition;
adding an aqueous liquid to the composition as a result of which a hydrolyzable mixture is formed;
thermally hydrolyzing the hydrolyzable mixture;
at least partially separating the solid and liquid phases from the thermally hydrolyzed mixture, resulting in a second mixture with a dry matter content that is less than the dry matter content of the thermally hydrolyzed mixture, and a third mixture with a higher dry matter content,
characterized in that the method further comprises a step of further enzymatically hydrolyzing the second and/or third mixture.

In a first step of the method, a composition comprising feathers and/or hair and water is provided, the composition having a dry matter content of less than 50 wt % with respect to the total weight of the composition.

According to certain embodiments of the present invention, the composition may be any by-product or waste material of biological origin, including in particular organic material, particularly derived from poultry, bovines, ovines, porcines, pets, birds, fur-bearing animals, and the like, usually obtained from an slaughterhouse or fat rendering plant (hereinafter referred to as “starting material”), preferably obtained from a poultry or pig slaughterhouse or poultry or pig fat rendering plant. Today, the common practice is that these by-products or waste materials of biological origin are poultry feathers or hair, transported by truck from a slaughterhouse to a conversion plant. According to an embodiment, the composition comprises feathers as the only solid product. According to an embodiment, the composition comprises hair, for example wool, as the only solid product. According to an embodiment, the composition comprises feathers and hair as the only solid products. According to an embodiment, the composition comprises animal hides.

Usually, the dry matter content of the starting material on arrival at the conversion plant is unknown due to the fact that the pre-treatments at the slaughtering plant are often an unknown factor. Consequently, upon arrival at the conversion plant, said starting material is preferably subjected to a pretreatment step. Said pretreatment step may comprise in particular, without being limited thereto: adding and/or removing water, cleaning, rinsing, sorting, degreasing, cutting, milling, grinding, or any combination thereof.

Such pretreatment step may facilitate the handling of the starting material such as to render the same more easily removable and transportable. It also removes unwanted organic and inorganic material, thereby increasing the quality of the final product. It may also further improve the efficiency of further processing steps.

Preferably, the starting material is supplied to a feeder system, which may comprise at least a large compartment such as a bunker for storing the starting material, and/or a washing and/or transport system. In the feeder system, the starting material can be mixed with water to obtain the composition of the process of the present invention.

Such a feeder system may advantageously provide at the same time for the transport of the starting material, as well as for washing and/or separating the starting material from unwanted organic material, such as blood, fat, or portions of the animal's body, and/or from unwanted inorganic material, such as plastic, metal parts, sand and rubber. Said feeder system may also advantageously allow to segregate thick packs of entangled starting material into smaller, transportable portions.

The feeder system, as mentioned above, typically uses commercially available devices, such as screw conveyors, pumps, and/or revolving or rotary screens. Impurities or undesired matter can be eliminated by means of sieves positioned below the screw conveyor channel. Alternatively the revolving screen has perforations on the walls that allow the elimination of impurities while tumbling.

According to an embodiment, the dry matter content is at most 40 wt %, more preferably at most 30 wt %, even more preferably at most 25 wt %.

According to one embodiment, the feathers and/or the hair absorb water at least partially.

When the starting material comprises an amount of water that exceeds the preferred amount of water, the washing and/or conveying system advantageously comprises a dewatering screw, with the aim of removing at least part of the water from the starting material to obtain the composition of the present invention.

In a second step, the invention provides for pressing at least part of the water of composition.

Preferably, the dry matter content of the composition after pressing is at least 32 wt %, more preferably at least 34 wt %, even more preferably at least 36 wt %, more preferably at least 38 wt %, and most preferably at least 40 wt % relative to the total weight of the composition.

It should further be clear that the dry matter content of the composition after pressing is preferably at most 58 wt %, more preferably at most 56 wt %, even more preferably at most 54 wt %, more preferably at most 52 wt %, and most preferably at most 50 wt % relative to the total weight of the composition.

According to an embodiment of the process according to the present invention, the act of draining the at least part of the water of the composition can be performed by using a force of gravity, in particular the pressing force of the feathers and/or hair which are present in the composition. More in particular, the feathers and/or hair which are present in the composition can be packed together in the form of at least one layer, wherein the feathers and/or hair in the top portion of the at least one layer exert pressure on the feathers and/or hair situated in the lower portions of the at least one layer, thereby draining at least part of the water which is at least partially absorbed by said feathers and/or hair. Alternatively, the at least one part of the water from the composition can be removed by means of a decanter.

In a preferred embodiment of the process according to the present invention, the act of pressing the at least one part of the water of the composition can be performed using a dewatering press, of which several models are available on the market. Due to the act of draining at least a part of the water, the feathers and/or hair are mechanically dewatered and macerated as they are forcibly conveyed through the press. A screw conveyor or screw press that continuously compresses the material may be used. Such a device comprises an elongated, cylindrical barrel housing containing an axially disposed rotatable shaft for driving a feed worm wheel to advance wet material through the barrel, and a plurality of knife bars protruding between the worm wheel flights for cooperating with the latter to effect a compression process. The press barrel is typically made of stainless steel cage bars, which capture, drain, and press feathers or hair.

Water is collected and squeezed out of the feathers and/or hair and may be drained through a sewage pipe located at the bottom, or the water can flow through slots in the compression barrel of the screw press. Preferably, the collected water is returned to the feeder system.

According to an alternative embodiment, dewatering can be carried out by thermal drying, for example by passing through jacketed, steam-heated vessels.

According to certain embodiments of the process according to the present invention, the composition after being pressed out is preferably ground before the third step.

Grinding of the composition after squeezing can be carried out with a grinding device, a mixer equipped with a shaft with one or more rotating discs, or any other device available on the market. Radial vanes on the disc's periphery may be used to blow out the feathers and hair present in the composition after draining at high velocity. The rotating discs have preferably a tooth pattern. These devices shred the wet feathers or wet hair present in the composition after draining. The advantage of grinding the feathers and/or hair before the hydrolysis is that the composition after draining will be a pulpier mixture, allowing easier hydrolysis at lower energy costs.

In a third step of the process of the present invention, an aqueous liquid is added to the composition after draining, as described above, to form the hydrolyzable mixture, the hydrolyzable mixture having a dry matter content of between 20 and 50 wt %.

According to a preferred embodiment of the process according to the present invention, said hydrolyzable mixture has a dry matter content of at most 48 wt %, preferably at most 46 wt %, more preferably at most 44 wt %, even more preferably at most 42 wt % and most preferably at most 40 wt %, relative to the total weight of the hydrolyzable mixture. It should further be understood that said hydrolyzable mixture has a dry matter content of at least 22 wt %, preferably at least 24 wt %, more preferably at least 26 wt %, even more preferably at least 28 wt % and most preferably at least 30 wt %, relative to the total weight of the hydrolyzable mixture.

According to certain embodiments of the process according to the present invention, the aqueous liquid is a liquid comprising water.

Preferably, the aqueous liquid has a temperature which is at least 50° C., more preferably at least 60° C., even more preferably at least 70° C., more preferably at least 80° C., and most preferably at least 85° C.

According to an embodiment, the aqueous liquid is a liquid comprising water, furthermore, enriched with at least amino acids, polypeptides and/or proteins, wherein in particular these proteins, polypeptides and/or amino acids are derived from feathers and/or hair.

According to a more preferred embodiment of the process according to the present invention, said aqueous liquid is an enriched proteinaceous liquid which is obtained as a product stream after hydrolyzing the hydrolysable mixture, as explained in detail below.

In a fourth step of the process of the present invention, the hydrolyzable mixture is thermally hydrolyzed.

According to an embodiment, the hydrolysis time of the hydrolyzable mixture of the present invention is at least 3 minutes, preferably at least 4 minutes, and most preferably at least 5 minutes. Furthermore, it should be clear that the hydrolysis time is at most 25 minutes, preferably at most 20, preferably at most 19 minutes, more preferably at most 18 minutes, even more preferably at most 17 minutes, more preferably at most 16 minutes, and most preferably at most 15 minutes. According to an embodiment, the thermal hydrolysis continues for a period between 10 sec and 4 hours, preferably between 1 min and 20 min, even more preferably between 5 and 15 min.

According to an embodiment, the hydrolysis can continue in one or more reactors. According to an embodiment, the at least one reactor is operated at an elevated pressure of at least 3 bar, preferably at least 4 bar, more preferably at least 5 bar, and most preferably at least 6 bar. Furthermore, it should be clear that the at least one reactor is advantageously used at an elevated pressure of at most 12 bar, preferably at most 11 bar, more preferably at most 10 bar. Advantageously, the at least one reactor is operated at a temperature of at least 135° C., preferably at least 145° C., more preferably at least 155° C., even more preferably at least 165° C., and most preferably at least 175° C. It is further understood that the at least one reactor is advantageously operated at a temperature of at most 245° C., preferably at most 235° C., more preferably at most 225° C., even more preferably at most 215° C., more preferably at most 205° C., and most preferably at most 195° C.

In a fifth step of the process of the present invention, at least part of the solid and liquid phases of the thermally hydrolyzed mixture are separated from each other, resulting in a second mixture with a dry matter content that is less than the dry matter content of the thermally hydrolyzed mixture, and a third mixture with a higher dry matter content,

The method of producing a hydrolyzed mixture of feathers and/or hair according to the present invention further comprises the step of separating the hydrolyzed mixture, as described in detail above, into i) at least one solid phase portion and ii) at least one liquid portion by means of suitable equipment. As typical suitable equipment mention may be made in particular of a press and a decanter and/or a centrifuge.

In the context of the invention, the term “liquid portion” refers to the supernatant remaining on top of the solid phase portion in the suitable equipment used for the separation.

According to an embodiment of the method according to the present invention, the dry matter content after removal of at least a part of the solid phase is between 5 and 30 wt %, i.e. of the second mixture. The dry matter content in the remaining fraction, the third mixture, is higher than 30 wt %.

According to an embodiment of the method, at least part of the at least one liquid portion is subjected to evaporation, whereby an enriched proteinaceous liquid is formed, with a dry matter content. Preferably, the dry matter content of the enriched liquid is at least 40 wt %, preferably at least 43 wt %, more preferably at least 45 wt %, and most preferably at least 48 wt %. Furthermore, it should be clear that the dry matter content of the enriched liquid is at most 60 wt %, preferably at most 57 wt %, and more preferably at most 54 wt %. It should be understood that the enriched liquid may be liquid at the vaporization temperature but may be a paste at room temperature.

In a sixth step of the process of the present invention, the second and/or third mixture is further enzymatically hydrolyzed.

Surprisingly, the inventors have now found that adding enzymes to the thermally hydrolyzed mixture results in an increase in the content of low molecular weight protein and/or amino acids and with bio stimulating properties. A biostimulant is a product that positively influences both the growth and the resistance of plants, preferably only limited quantities are needed for this. Formed D-amino acids also increase the effectiveness as a biostimulant and do not pose a problem

Because the mixture is thermally hydrolyzed before the enzymatic hydrolysis, the structure is already partially broken down and opened. As a result, D-amino acids do not get in the way and enzymes can better break down the structure further. This process step reduces the energy and time required and results in better operation. Because this thermal hydrolysis takes place in an aqueous environment, no special chemicals, acids or bases are required, nor a neutralization step. The thermal hydrolysis ensures a reduced cost over the entire process. Less expensive enzymes must be used to carry out the reaction. In addition, inhibitors are removed from the mixture during thermal hydrolysis and/or during other steps. The mixture that is enzymatically hydrolyzed will also be rendered completely sterile by the thermal hydrolysis, preventing unwanted (bacterial) reactions that can influence the characteristics of the end product in an undesired and/or uncontrolled manner. Therefore, this thermal hydrolysis step is essential for a good process.

According to an embodiment, endoproteases are used. According to an embodiment, exoproteases are used. According to an embodiment, exoproteases and endoproteases are used. According to an embodiment, the enzymatic hydrolysis takes place at a temperature higher than 40° C., preferably between 4° and 80° C., more preferably between 5° and 70° C. and even more preferably between 5° and 55° C. According to an embodiment, the enzymatic hydrolysis takes place for a period of at least 10 minutes and preferably between 10 minutes and 12 hours, more preferably between 3 hours and 7 hours, even more preferably between 5 hours and 6 hours. According to an embodiment, the enzymatic hydrolysis takes place at a pH of 5 to 9, preferably between 6 and 8, preferably about 7.2. According to an embodiment, the water-to-protein ratio during enzymatic hydrolysis is between 50:1 and 1:1, preferably between 20:1 and 1:1. These parameters were found to be optimal for obtaining a mixture as a biostimulant. An advantage over acid hydrolysis is that, for example, no neutralization is required afterwards, and fewer salts are present. According to an embodiment, no strong acids or bases are added before or during the hydrolysis.

According to an embodiment of the method according to the present invention, disulphide-breaking components such as dithiothreitol, Na₂S, NaHS are present in the second mixture. These components support the hydrolysis. According to an embodiment of the method according to the present invention, Na₂S is used in a concentration of at least 1 g/L, preferably at least 3 g/L and at most 10 g/L, preferably at most 7 g/L.

According to an embodiment of the method according to the present invention, the method further comprises a step of ultrafiltration, reverse osmosis and/or drying.

According to an embodiment, at least part of the enzymatically hydrolyzed mixture is dried. Preferably, drying of at least part of the solid phase portion is suitably carried out on a band or belt dryer, a fluidized bed dryer, or a conveyor dryer. Preferably, the drying is carried out with air, the solid phase portion having a temperature of at most 80° C., preferably at most 75° C. and most preferably at most 70° C. According to an embodiment, the drying is carried out under atmospheric pressure, preferably at a negative pressure relative to the atmospheric pressure of −20 to −40 mbar, more preferably at a negative pressure of about −30 mbar.

According to an embodiment, at least part of the enzymatically hydrolyzed mixture is subjected to ultrafiltration. The pressure during ultrafiltration is 2 to 10 bar and the membrane size is 2 nm to 0.1 μm. According to an embodiment, the pressure is between 2 and 5 bar and the membrane size is between 2 and 50 nm. According to an embodiment, the pressure is between 5 and 10 bar and the membrane size is between 50 and 100 nm.

According to an embodiment, at least part of the enzymatically hydrolyzed mixture is subjected to reverse osmosis. The pressure during reverse osmosis is 10 to 100 bar and the membrane size is 0.1 nm to 1 nm. According to an embodiment, the pressure is between 10 and 50 bar and the membrane size is between 0.1 and 0.50 nm. According to an embodiment, the pressure is between 50 and 100 bar and the membrane size is between 0.5 and 1.0 nm.

According to an embodiment, at least part of the enzymatically hydrolyzed mixture is subjected to a combination of at least two techniques including ultrafiltration, reverse osmosis and drying.

According to an embodiment of the method, the method further comprises a step of adding sulfur-binding components, preferably Cu₂(OH)₃Cl, ZnSO₄ of CuSO₄. These components ensure a reduction of the volatile sulfur components that can cause an undesirable odor.

According to another embodiment of the method, at least part of the liquid portion is used to form the aqueous liquid that is added to the composition, thereby forming the hydrolyzable mixture as described above.

According to an alternative embodiment of the method, the hydrolyzed mixture is mixed with at least part of the enriched liquid.

According to an embodiment, the hydrolyzed mixture is mixed with an acid. Because an acid is added, the mixture will remain stable for a longer period of time.

In an embodiment, an aqueous liquid comprising proteins and/or amino acids is added to the composition. This results in an increase in the protein and/or amino acid content of the hydrolyzable mixture. As such, the protein and/or amino acids content of the final feather meal product can also be influenced.

The combination between adding water, thermal hydrolysis and subsequent enzymatic hydrolysis is very important for proper degradation of feathers and/or hair. These structures are difficult to break down into small proteins suitable for use as a biostimulant.

In a second aspect, the invention relates to an enzymatically hydrolyzed mixture obtained according to the method according to the previous aspect, as described above.

According to a preferred embodiment, more than 40 wt % of the peptides in the enzymatically hydrolyzed mixture have a molecular mass of less than 2500 Dalton, preferably more than 50 wt %, more preferably more than 70 wt %, even more preferably more than 80 wt %. According to a preferred embodiment, more than 20 wt % of the peptides in the enzymatically hydrolyzed mixture have a molecular mass of less than 1000 Dalton, preferably more than 30 wt %, more preferably 50 wt %, even more preferably more than 60 wt %.

According to an embodiment, 40-99 wt % of the peptides in the enzymatically hydrolyzed mixture have a molecular mass below 2500 Dalton, preferably 50-99 wt %, more preferably 70-99 wt %, even more preferably 80-99 wt %. According to an embodiment, 20-99 wt % of the peptides in the enzymatically hydrolyzed mixture have a molecular mass below 1000 Dalton, preferably 30-99 wt %, more preferably 50-99 wt %, even more preferably 60-99 wt %. According to an embodiment, 0.005-5 wt % of the peptides in the enzymatically hydrolyzed mixture have a molecular mass greater than 300000 Dalton, preferably 0.01-2 wt %, more preferably 0.1-1.5 wt %. This is in contrast to the peptides in the mixture that was only thermally treated. In a mixture that was only thermally treated, 10-15 wt % of the peptides have a molecular mass higher than 300000 Dalton. The hydrolysates described in BE1024444 contain a large amount of heavy peptides which may be less easily absorbed.

According to an embodiment, 20-80 wt % of the peptides in the enzymatically hydrolyzed mixture have a molecular mass below 1000 Dalton, preferably 30-70 wt %, more preferably 40-60 wt %, even more preferably 45-55 wt %. According to an embodiment, 10-50 wt % of the peptides in the enzymatically hydrolyzed mixture have a molecular mass between 1000 and 5000 Dalton, preferably 15-45 wt %, more preferably 20-40 wt %, even more preferably 25-35 wt %. According to an embodiment, 5-50 wt % of the peptides in the enzymatically hydrolyzed mixture have a molecular mass between 5000 and 10000 Dalton, preferably 10-40 wt %, more preferably 10-30 wt %, even more preferably 15-25 wt %.

According to an embodiment, 40-60 wt % of the peptides in the enzymatically hydrolyzed mixture have a molecular mass below 1000 Dalton, 20-40 wt % of the peptides in the enzymatically hydrolyzed mixture have a molecular mass between 1000 and 5000 Dalton and 10-30 wt % of the peptides in the enzymatically hydrolyzed mixture have a molecular mass between 5000 and 10000 Dalton.

According to an embodiment, the enzymatically hydrolyzed mixture comprises 0.5-4 times more peptides with a molecular mass lower than 1000 Dalton than peptides with a molecular mass higher than 1000 Dalton, preferably 0.7-2 times more, more preferably 0.7-1.3 times more. According to an embodiment, the enzymatically hydrolyzed mixture comprises 1-8 times more peptides with a molecular mass lower than 5000 Dalton than peptides with a molecular mass higher than 5000 Dalton, preferably 2-6 times more, more preferably 2-4 times more. According to an embodiment, the average molecular mass of the peptides in the enzymatically hydrolyzed mixture is 1000-3000 Da, preferably 1500-3000 Da.

According to an embodiment, the enzymatically hydrolyzed mixture comprises 20-30 wt % proteins, 2-7 wt % fats, 0.5-4 wt % salts and 45-70 wt % moisture. According to an embodiment, the enzymatically hydrolyzed mixture comprises 75-95 wt % proteins, 2-12 wt % fats, 0.5-4 wt % salts and 3-10 wt % moisture. According to an embodiment, the enzymatically hydrolyzed mixture comprises 3-15 times more by weight of proteins than fats, preferably 4-7 times more. According to an embodiment, the enzymatically hydrolyzed mixture comprises 8-25 times more by weight of proteins than salts, preferably 12-15 times more.

According to an embodiment, the enzymatically hydrolyzed mixture comprises 77-100 wt % of levorotatory amino acids relative to the total number of free amino acids, preferably 80-95%, more preferably 85-95%.

The term “digestible protein” as used in this document, refers to the proteins capable of being digested by enzymes in an animal's digestive system, as measured by pepsin digestibility. This is a subset of the total protein content.

According to a preferred embodiment, an enzymatically hydrolyzed mixture comprises at least 5 wt %, preferably at least 7 wt %, more preferably at least 8 wt % protein with respect to the total weight. At least 80 wt %, preferably at least 85 wt %, more preferably at least 90 wt %, even more preferably at least 95 wt % of these proteins are digestible proteins.

According to a preferred embodiment, the enzymatically hydrolyzed mixture consists of a high percentage of cysteine (3 wt % of the protein) and serine (8.5 wt % of the protein). The relative amount of Phenylalanine also increases to more than 3 wt %, preferably 4 wt %, during thermal and enzymatic hydrolysis. In addition, the enzymatically hydrolyzed mixture also contains a lot of sulfur (min 1.5 wt % relative to dry matter).

According to an embodiment, 2-8 wt % of the amino acids of the enzymatically hydrolyzed mixture is cysteine, preferably 2-5 wt %. According to an embodiment, 4-12 wt % of the amino acids of the enzymatically hydrolyzed mixture is serine, preferably 6-10 wt %. According to an embodiment, 2-8 wt % of the amino acids of the enzymatically hydrolyzed mixture is phenylalanine, preferably 2-6 wt %. According to an embodiment, 2-8 wt % of the amino acids of the enzymatically hydrolyzed mixture is tryptophan, preferably 2-5 wt %. According to an embodiment, 4-12 wt % of the amino acids of the enzymatically hydrolyzed mixture is glycine, preferably 6-10 wt %. According to an embodiment, the enzymatically hydrolyzed mixture comprises 1-15 wt % sulfur relative to dry matter, preferably 1.5-10 wt %. According to an embodiment, the proteins present in the composition comprise 0.7-1.2 times more tryptophan than cysteine. According to an embodiment, the proteins present in the composition comprise 3-5 times more serine than cysteine. According to an embodiment, the proteins present in the composition comprise 3-9 times more glutamic acid than cysteine.

According to an embodiment of the invention, the composition additionally contains one or more pesticides. In further embodiments of the invention, the composition further contains one or more surfactants. Furthermore, the composition may contain one or more UV-filtering substances. In a further embodiment, the composition further contains an anti-foaming agent. In a further embodiment, the composition further contains a fertilizer. Preferably, the composition is diluted with water.

In the following aspects, the invention relates to the use of the enzymatically hydrolyzed mixture, the thermally hydrolyzed mixture, the second or third mixture, as described above, as a biostimulant. Preferably, the use of the mixture described herein comprises an increase in the crop yield of the plant. It is clear that this is a particular advantage for the agricultural industry, which strives to optimize crop yields in respect of the resources used. Crop yield optimization has so far only been successfully achieved through the use of chemical compounds that have proven to be unsafe for human health, and/or that pose a significant burden to the environment. Therefore, using the current composition is safe to use while efficiently optimizing crop yield in a targeted plant.

According to an embodiment, a mixture of the enzymatically hydrolyzed mixture, the thermally hydrolyzed mixture, the second and/or third mixture, as described above, is used as a biostimulant.

According to an embodiment, the stimulation of plant growth comprises increasing the flowering of said target plant. While the stimulation of harvest is a particular benefit, some crops are grown specifically for their flowers. It has been found that the use of the composition as described herein results in the increase of flowering, which is of particular importance to the floriculture industry. As such, use of the composition will result in a greater number of flowers per plant and/or will successfully stimulate the growth of the plant toward a higher ratio of flowers to other plant parts, such as foliage, stems, roots, etc.

In some embodiments, the stimulation of plant growth comprises shortening the time to fruit harvest. Using the composition directs energy use in the plant towards generative growth, thereby increasing the efficiency of fruit growth, resulting in a shorter time to harvest.

In some embodiments, stimulating plant growth comprises increasing the amount of fruit produced by the target plant. While a general boost to crop yield is a particular benefit, some crops are grown specifically to harvest fruit, e.g. tomato plants. The use of the current composition now allows for a greater amount of fruit produced per plant, thus making it possible to harvest a greater amount of fruit on the same area of land, or alternatively harvest the same amount of fruit on a smaller area of land.

According to an embodiment, the mixture is applied to the crops by spraying or misting. By spraying or misting, a uniform and even distribution of the composition on the crops can be obtained. According to a preferred embodiment, the enzymatically hydrolyzed mixture is atomized.

According to an embodiment, the total amount when applied to crops is at least 0.25 L/hectare, preferably 5 L/hectare, more preferably 8 L/hectare and most preferably 10 L/hectare and at most 25 L/hectare, preferably 20 L/hectare, more preferably 18 L/hectare and most preferably 15 L/hectare. As understood by those skilled in the art, this amount is expressed per growing season and this amount is preferably reapplied at least annually. According to an embodiment, the amount is applied at different times with a period between two applications of at least 1 week, preferably 1 month. According to an embodiment, the amount is applied to a plant at 2, 3, 4 or 5 different times. According to an embodiment, 3 L/hectare is applied 4 times during a growing season. According to an embodiment, 4 L/hectare is applied 3 times during a growing season.

According to an embodiment, the amount of protein after evaporation is 300 g/L. This means about 45-50 g nitrogen/L. An average dose of 15 liters/ha as a biostimulant via foliar spraying over an entire growing season therefore means an addition of 0.7 kg N/ha. It is therefore proposed to apply an amount of nitrogen that is many times lower than the nitrogen deposition from the air in Western European countries. In 2018, the average nitrogen deposition in Flanders was 23.2 kg N/ha. It is clear that the effect of applying this protein mixture is not based on nitrogen addition but stimulates the growth of the plant in a much more efficient way.

The use of the enzymatically hydrolyzed mixture, even in low concentrations, reduces the need for traditional fertilization. With traditional fertilization, a large amount of nutrients is sprayed onto or injected into the soil. Only a limited amount of this is absorbed by the plant, which leads to ecological problems. By spraying low amounts of the enzymatically hydrolyzed mixture on the plants, the nitrogen load on the environment is reduced. In this way, nitrogen-polluted soils can slowly recover.

Without wanting to be limited to theory, the hydrolyzed mixture ensures that the plant saves a lot of energy. The enzymatically hydrolyzed mixture contains amino acids in “free state” or as short peptides. By absorbing these, plants do not have to synthesize them themselves.

Preferably, plants are chosen from the group of annual and perennial mono- or dicotyledon crops. The composition according to the present invention can be used on crops that are monocotyledonous as well as on dicotyledonous crops and shows a good effect on both groups of plants. Preferably, the crops are selected from fruit trees, cereal plants, oilseed rape, beets, potatoes, and possible combinations thereof. These agricultural crops show a strongly increased yield after application of the composition according to the present invention.

This invention further relates to a use as described above wherein influencing the life processes of crops is selected from increasing the yield of crops, increasing the fruit setting of crops or the number of fruits or seeds, increasing the fruit or grain size of crops, the fruit size or diameter of fruits or seeds, improving the color or coloring of the fruits, influencing the ripening of crops, increasing the protein or protein content of fruits or seeds, increasing of the total yield, increasing the yield of a first pick, increasing the yield of colored fruit on a first pick, shortening the elongation of shoots, and combinations thereof.

The invention relates to the use of a hydrolyzed mixture as a biostimulant, wherein the average molecular mass of the peptides in the hydrolyzed mixture is 1000-3000 Da, preferably 1500-3000 Da. According to an embodiment, the hydrolyzate comprises 50-90 wt % peptides on a dry matter basis. According to an embodiment, the hydrolyzate comprises 4-7 times more peptides than fats, preferably 5-6 times more. According to an embodiment, 0.2-1.2 kg N/ha is applied when using this biostimulant, preferably 0.5-0.9, more preferably about 0.7.

In what follows, the invention is described by way of non-limiting examples illustrating the invention, and which are not intended to or should not be interpreted as limiting the scope of the invention.

### EXAMPLES

The invention will now be further explained on the basis of the following example, without however being limited to this.

### Example 1

Example 1 concerns a comparison between a thermally hydrolyzed mixture (Product 1) and hydrolyzed mixtures obtained according to the method according to the present invention (Product 2 & Product 3).

Table 1 shows the weight distributions of molecular weight (in Daltons). This comparison shows that the additional enzymatic hydrolysis has greatly increased the molecular weight. For Product 1, the molecular weight is >2000 Dalton for 59.54 wt %. For Products 2 and 3 this is 23.95 and 21.10 wt %, respectively. For Products 2 and 3, the center of gravity of their molecular weight distribution is below 1000 Dalton, at 51.86 and 56.07 wt % respectively. By comparison, only 25.69 wt % of Product 1 has a molecular weight below 1000 Dalton.

TABLE 1
molecular weight distribution of 3 products, whereby
Product 1 has been thermally hydrolyzed and Product
2 and 3 thermally and enzymatically.

| Mol. Weight | Product 1 | Product 2 | Product 3 |
|---|---|---|---|
| (Dalton) | (wt %) | (wt %) | (wt %) |

| | | | | |
|---|---|---|---|---|
| > | 300000 | 12.11 | 0.2 | 0.9 |
| 30000 | 25000 | 2.02 | 0.12 | 0.14 |
| 25000 | 20000 | 2.58 | 0.22 | 0.24 |
| 20000 | 15000 | 3.68 | 0.46 | 0.45 |
| 15000 | 10000 | 5.88 | 1.19 | 1.05 |
| 10000 | 7500 | 4.7 | 1.42 | 1.23 |
| 7500 | 5000 | 7.62 | 3.2 | 2.74 |
| 5000 | 2500 | 15.62 | 11.21 | 9.34 |
| 2500 | 2000 | 5.33 | 5.93 | 5.01 |
| 2000 | 1900 | 1.23 | 1.54 | 1.35 |
| 1900 | 1800 | 1.27 | 1.7 | 1.49 |
| 1800 | 1700 | 1.32 | 1.78 | 1.57 |
| 1700 | 1600 | 1.33 | 1.9 | 1.69 |
| 1600 | 1500 | 1.41 | 2.13 | 1.92 |
| 1500 | 1400 | 1.43 | 2.3 | 2.1 |
| 1400 | 1300 | 1.5 | 2.62 | 2.42 |
| 1300 | 1200 | 1.58 | 3.23 | 3.05 |
| 1200 | 1100 | 1.81 | 3.53 | 3.54 |
| 1100 | 1000 | 1.89 | 3.46 | 3.7 |
| 1000 | 900 | 1.79 | 3.74 | 4.38 |
| 900 | 800 | 1.77 | 4.27 | 5.37 |
| 800 | 700 | 1.83 | 4.84 | 6.13 |
| 700 | 600 | 2.01 | 5.63 | 6.97 |
| 600 | 500 | 2.23 | 5.7 | 8.03 |
| 500 | 400 | 2.15 | 4.53 | 6.73 |
| 400 | 300 | 3.87 | 9.00 | 8.15 |
| 300 | 204 | 5.09 | 7.84 | 5.4 |
| 204 | < | 4.95 | 6.31 | 4.91 |

| | | | |
|---|---|---|---|
| Total | 100 | 100 | 100 |

### Example 2

This example concerns a comparison in the amino acid composition of the feathers compared to that of Product 2 from Example 1. The different amino acids were reported based on the total protein present in the product. The comparison in amino acid composition between the initial and end product shows that Phenylalanine and Cysteine in particular increase relatively (Table 2). Especially Alanine, Methionine and Aspartate decrease in relative quantity.

| Amino acids | Feathers | Product 2 |
|---|---|---|
| Arginine | 6.27 | 7.03 |
| Lysine | 2.59 | 2.14 |
| Alanine | 6.06 | 4.54 |
| Threonine | 4.32 | 4.78 |
| Glycine | 9.21 | 7.78 |
| Valine | 6.34 | 7.58 |
| Serine | 9.83 | 10.73 |
| Proline | 10.20 | 11.13 |
| Isoleucine | 4.14 | 4.78 |
| Leucine | 7.05 | 8.03 |
| Methionine | 0.86 | 0.65 |
| Histidine | 0.85 | 0.71 |
| Phenylalanine | 0.81 | 4.87 |
| Glutamic acid | 13.90 | 11.2 |
| Aspartate | 8.04 | 6.18 |
| Cysteine | 2.21 | 4.28 |
| Tyrosine | 2.39 | 2.92 |
| Tryptophan | 0.41 | 0.66 |

1. A method of producing an enzymatically hydrolyzed mixture of feathers and/or hair and/or skins, said method comprising:
providing a composition comprising feathers and/or hair and water, the composition having a dry matter content of less than 50 wt % with respect to the total weight of the composition;
pressing at least a part of the water from the composition;
adding an aqueous liquid to the composition as a result of which a hydrolyzable mixture is formed;
thermally hydrolyzing the hydrolyzable mixture;
at least partially separating solid and liquid phases from the thermally hydrolyzed mixture, as a result of which a second mixture is obtained with a dry matter content that is less than the dry matter content of the thermally hydrolyzed mixture, and a third mixture with a higher dry matter content,
characterized in that the method further comprises enzymatically hydrolyzing the second and/or third mixture.

2. The method according to claim 1, characterized in that the enzymatically hydrolyzing takes place at a temperature between 4° and 80° C.

3. The method according to claim 1, characterized in that the enzymatically hydrolyzing takes place for a period between 10 minutes and 12 hours.

4. The method according to claim 1, characterized in that disulphide-breaking components are present in the second mixture.

5. The method according to claim 1, characterized in that the method further comprises ultrafiltration, reverse osmosis and/or drying.

6. The method according to claim 1, characterized in that the method further comprises a step of adding sulfur-binding components.

7. The method according to claim 1, characterized in that the thermally hydrolyzing takes place for a period between 10 sec and 4 hours.

8. The method according to claim 1, characterized in that the dry matter content after removal of at least a solid phase part is between 5 and 30 wt %.

9. An enzymatically hydrolyzed mixture obtained according to the method according to claim 1.

10. The enzymatically hydrolyzed mixture according to claim 9, characterized in that more than 40 wt % of peptides have a molecular mass below 2500 Dalton.

11. The enzymatically hydrolyzed mixture according to claim 10, characterized in that more than 20 wt % of the peptides have a molecular mass below 1000 Dalton.

12. A use of the enzymatically hydrolyzed mixture according to claim 9, as a biostimulant.

13. A use of the thermally hydrolyzed mixture, the second or third mixture, obtained according to the method according to claim 1, as a biostimulant.

14. The use according to claim 12, characterized in that the enzymatically hydrolyzed mixture is applied by spraying or misting.

15. The use according to claim 12, characterized in that the total amount when applied to crops is at least 0.25 L/hectare and at most 25 L/hectare.

16. The method according to claim 1, characterized in that the thermally hydrolyzing is carried out at a temperature of at least 175° C.

17. The method according to claim 1, characterized in that the thermally hydrolyzing is carried out at a pressure of at least 6 bar.

18. The method according to claim 1, characterized in that the thermally hydrolyzing is carried out at a temperature of at least 175° C. and a pressure of at least 6 bar.

19. The enzymatically hydrolyzed mixture according to claim 9, wherein 20-80 wt % of the peptides in the enzymatically hydrolyzed mixture have a molecular mass below 1000 Dalton, wherein 10-50 wt % of the peptides in the enzymatically hydrolyzed mixture have a molecular mass between 1000 and 5000 Dalton, and wherein 5-50 wt % of the peptides in the enzymatically hydrolyzed mixture have a molecular mass between 5000 and 10000 Dalton.
